(19) United States
(12) Patent Application Publication
(10) Pub. No.: US 2026/0165878 A1
(43) Pub. Date: Jun. 18, 2026

(54) COLD COMPRESS BAG AND COLD COMPRESS SYSTEM COMPRISING SAME

(71) Applicant: Ningbo KK-Bioscience Co., Ltd., Ningbo City (CN)

(72) Inventor: Xingwen XIE, Ningbo City (CN)

(21) Appl. No.: 19/421,450

(22) Filed: Dec. 16, 2025

(30) Foreign Application Priority Data
Dec. 18, 2024 (CN) 202411865730.8

(51) Int. Cl. A61F 7/10; A61F 7/02

(52) U.S. Cl. CPC A61F 7/10; A61F 2007/0244; A61F 2007/0273; A61F 2007/108

(57) ABSTRACT

The present disclosure provides a cold compress bag and a cold compress system including the same. The cold compress bag includes a first surface layer, an intermediate layer, a second surface layer, and a partition assembly. A liquid storage space is defined between the first surface layer and the intermediate layer. An air storage space is defined between the second surface layer and the intermediate layer. The air storage space is provided with a vent port configured to communicate the air storage space with outside. The partition assembly is configured to connect to the first surface layer and the intermediate layer to divide the liquid storage space into a liquid inlet region, and a liquid outlet region in communication with the liquid inlet region. The liquid inlet region is provided with a liquid inlet port configured to communicate the liquid inlet region with outside.

### CROSS REFERENCE TO THE RELATED APPLICATION

This application is based upon and claims priority to Chinese Patent Application No. 202411865730.8, filed on Dec. 18, 2024, the entire contents of which are incorporated herein by reference.

### TECHNICAL FIELD

The present disclosure relates to the technical field of medical treatment, and more particularly to a cold compress bag and a cold compress system including the same.

### BACKGROUND

Cold therapy uses substances lower than the temperature of the human body to act on the surface of the human body to contract the blood vessels in skin and internal organs through nerve conduction, so as to change the circulation and metabolism of body fluids in various systems of the human body to achieve therapeutic purposes. In the medical field, cold therapy, as a physical therapy method, can alleviate local bleeding, edema, pain, and spasm, and has been widely used to treat various diseases and injuries.

Patients are often in need of bed rest after surgery. However, lack of activity will lead to slowed venous blood flow in the lower limbs, which increases the risk of thrombosis. Blood loss may occur during the surgical operation, which irritates the clotting system and makes it easier for the blood to clot, further increasing the risk of thrombosis. Cold compress bags in the related art can be used for cold therapy, but have little effect on preventing venous thrombosis after surgery.

### SUMMARY

An objective of the present disclosure is to provide a cold compress bag, to provide a cold therapy effect and prevent venous thrombosis.

Another objective of the present disclosure is to provide a cold compress system including the cold compress bag described above, to provide a cold therapy effect and prevent venous thrombosis.

To achieve at least one of the above objectives, the following technical solution is adopted in the present disclosure. A cold compress bag is provided, including: a first surface layer, an intermediate layer, and a second surface layer, where a liquid storage space is defined between the first surface layer and the intermediate layer, an air storage space is defined between the second surface layer and the intermediate layer, the air storage space is provided with a vent port, and the vent port is configured to communicate the air storage space with outside; and a partition assembly, where the partition assembly is configured to connect to the first surface layer and the intermediate layer to divide the liquid storage space into a liquid inlet region, and a liquid outlet region in communication with the liquid inlet region, the liquid inlet region is provided with a liquid inlet port configured to communicate the liquid inlet region with the outside, and the liquid outlet region is provided with a liquid outlet port configured to communicate the liquid outlet region with the outside.

Preferably, the cold compress bag further includes: a plurality of guide portions, where the plurality of guide portions are configured to connect to the second surface layer and the intermediate layer to divide the air storage space along a first direction into a first clamping region, a retaining region, and a second clamping region in communication with one another.

Preferably, the plurality of guide portions are configured to connect to the first surface layer and the intermediate layer, the plurality of guide portions are spaced apart from an outer edge of the cold compress bag to define an outer passage between the plurality of guide portions and the outer edge of the cold compress bag, and the plurality of guide portions are spaced apart from the partition assembly to define an inner passage between the plurality of guide portions and the partition assembly.

Preferably, the partition assembly includes a primary partition and a secondary partition, the primary partition is configured to divide the liquid storage space along a second direction into the liquid inlet region and the liquid outlet region, the secondary partition is connected to the primary partition, an extending direction of the secondary partition forms an angle relative to an extending direction of the primary partition to define a transition passage on a side away from the liquid inlet port and the liquid outlet port, and the transition passage is configured to communicate the liquid inlet region with the liquid outlet region.

Preferably, the primary partition includes a first segment, a transition segment, and a second segment, the first segment is staggered with the second segment in the second direction; the transition segment extends in a curved manner and is configured to connect to the first segment and the second segment; and from a side adjacent to the liquid inlet port to a side away from the liquid inlet port in the first direction, a width of at least a part of the liquid inlet region in the second direction gradually increases.

Preferably, the secondary partition extends in a curved manner in the second direction, and a center of curvature of the secondary partition is located on a side facing the liquid inlet port and the liquid outlet port.

Preferably, the plurality of guide portions include a first guide portion and a second guide portion, the first guide portion and the second guide portion extend in a curved manner in the second direction, a center of curvature of the first guide portion and a center of curvature of the second guide portion are each located on a side facing the liquid inlet port and the liquid outlet port, the first guide portion is arranged closer to the liquid inlet port and the liquid outlet port than the second guide portion is, and a length of the first guide portion is greater than a length of the second guide portion.

Preferably, the cold compress bag further includes a plurality of shunt portions, where at least a subset of the plurality of shunt portions are arranged in an array to shunt a liquid in the liquid storage space.

Preferably, the outer edge of the cold compress bag includes a plurality of arc segments smoothly connected, the plurality of arc segments are configured to guide a flow direction of a liquid in the liquid storage space, the plurality of arc segments include a concave segment, the concave segment is curved inwardly toward a center of the cold compress bag, and when the cold compress bag is bound to a limb, the concave segment is configured to avoid a joint.

To achieve at least one of the above objectives, the following technical solution is adopted in the present disclosure. A cold compress system is provided, including: the cold compress bag according to any one of the above embodiments; and a cold compress device, including: a box body, where the box body is provided with an accommodating cavity configured to accommodate liquid; a box lid, where the box lid is configured to cover a top of the box body, the box lid includes a lid body, a liquid intake and drainage module, a pressurizing module, and a control module, and the lid body is provided with an accommodating space configured to accommodate the liquid intake and drainage module, the pressurizing module, and the control module; the liquid intake and drainage module is configured to circulate the liquid between the box body and the cold compress bag to allow the cold compress device to operate in a cold therapy mode; the liquid intake and drainage module is configured to evacuate the liquid from the cold compress bag to allow the cold compress device to operate in a drainage mode; the pressurizing module is configured to inflate the cold compress bag to allow the cold compress device to operate in a pressurizing mode; and the control module is configured to control the liquid intake and drainage module and the pressurizing module to allow the cold compress device to operate in at least one of the pressurizing mode, the cold therapy mode, and the drainage mode; and a liquid diversion structure, where the liquid diversion structure extends from the lid body to a bottom of the box body, and the liquid diversion structure is configured to communicate the accommodating cavity with the liquid intake and drainage module.

Compared with the prior art, the present disclosure has the following advantages.
(1) The air storage space of the cold compress bag may be inflated through the vent port, so that when the cold compress bag is bound to a limb of a patient, the cold compress bag can provide pneumatic compression therapy to prevent venous thrombosis.
(2) The liquid sequentially passes through the liquid inlet region and the liquid outlet region through the liquid inlet port and the liquid outlet port, and circulates between the cold compress bag and the cold compress device, to uniformly and continuously cold compress the limb of the patient.

### BRIEF DESCRIPTION OF THE DRAWINGS

FIG. 1 is a schematic front view of a cold compress bag according to some embodiments of the present disclosure.

FIG. 2 is a schematic cross-sectional view of a cold compress bag according to some embodiments of the present disclosure.

FIG. 3 is a schematic front view of a cold compress bag according to some other embodiments of the present disclosure.

FIG. 4 is a schematic front view of a cold compress bag according to some other embodiments of the present disclosure.

FIG. 5 is a schematic rear view of a cold compress bag according to some embodiments of the present disclosure.

FIG. 6 is a schematic view showing the flow of liquid in a cold compress bag according to some embodiments of the present disclosure.

FIG. 7 is a schematic view of a cold compress system according to some embodiments of the present disclosure.

FIG. 8 is a schematic structural perspective view of a cold compress device according to some embodiments of the present disclosure.

FIG. 9 is a schematic view showing an interior of a box lid of a cold compress device according to some embodiments of the present disclosure.

FIG. 10 is a schematic cross-sectional view of a cold compress device according to some embodiments of the present disclosure.

FIG. 11 is a schematic structural perspective view of a box lid and a liquid diversion structure according to some embodiments of the present disclosure.

FIG. 12 is a schematic structural perspective view of a lower lid and a liquid diversion structure which are integrally formed according to some embodiments of the present disclosure.

FIG. 13 is a schematic structural perspective view of a pipe assembly according to some embodiments of the present disclosure.

In the drawings, the reference numerals respectively represent: 1. cold compress bag; 11. first surface layer; 12. intermediate layer; 13. second surface layer; 20. liquid storage space; 21. liquid inlet region; 211. liquid inlet port; 212. liquid inlet chamber; 22. liquid outlet region; 221. liquid outlet port; 222. liquid outlet chamber; 23. transition passage; 24. inner passage; 25. outer passage; 30. air storage space; 31. vent port; 32. first clamping region; 33. retaining region; 34. second clamping region; 40. partition assembly; 41. primary partition; 411. first segment; 412. transition segment; 413. second segment; 42. secondary partition; 50. guide portion; 51. first guide portion; 52. second guide portion; 60. shunt portion; 70. outer edge; 71. first convex segment; 72. concave segment; 73. second convex segment; 2. cold compress device; 80. box body; 81. accommodating cavity; 82. outer shell; 83. inner shell; 84. isolation spacing; 90. box lid; 91. lid body; 911. upper lid; 912. lower lid; 913. accommodating space; 92. liquid intake and drainage module; 93. pressurizing module; 94. control module; 101. liquid diversion structure; 1011. main body; 1012. outer arc portion; 1013. inlet hole; 1014. outlet hole; 201. pipe assembly; 2011. plate; 2012. primary pipe; 2013. secondary pipe; 2014. reinforcing rib; 2015. liquid diversion spacing; 301. filter member; 302. lighting lamp; 303. handle.

### DETAILED DESCRIPTION OF THE EMBODIMENTS

The present disclosure will be further described below through specific embodiments. It should be noted that the embodiments or technical features described below may be arbitrarily combined to form new embodiments without conflict.

In the description of the present disclosure, it should be noted that orientation and position relationships indicated by the orientation terms such as “center”, “transverse”, “longitudinal”, “length”, “width”, “thickness”, “on”, “below”, “front”, “rear”, “left”, “right”, “vertical”, “horizontal”, “top”, “bottom”, “inner”, “outer”, “clockwise”, and “counterclockwise” are based on the orientation or positional relationships shown in the drawings, and are only for the convenience of describing the present disclosure and simplifying the description, rather than indicating or implying that the apparatus or element described must have a specific orientation or be constructed and operated in a specific orientation, and therefore are not to be construed as limiting the present disclosure.

It should be noted that in the specification and claims of the present disclosure, the terms such as “first” and “second” are intended to distinguish between similar objects but do not indicate a particular order or sequence.

In the specification and claims of the present disclosure, the terms “include,” “comprise,” and any other variants thereof are intended to cover a non-exclusive inclusion. For example, a process, method, system, product, or device that includes a list of steps or units is not necessarily limited to those expressly listed steps or units, but may include other steps or units not expressly listed or inherent to such a process, method, product, or device.

It should be noted that, as used in the present disclosure, the terms such as “substantially,” “approximately,” and the like are used as terms indicating approximation, not as terms indicating degree, and are intended to indicate inherent deviations in measured or calculated values that would be recognized by those of ordinary skill in the art.

In the description of the present disclosure, it should also be noted that, unless otherwise clearly specified and defined, the terms such as “arrange”, “mount”, “connect”, “couple”, and variants thereof should be interpreted in a broad sense, for example, may be a fixed connection, a detachable connection, or an integral connection; may be a mechanical connection or an electrical connection; or may be a direct connection, a contact connection, an indirectly connection via an intermediate medium, or communication between the interiors of two components. For those of ordinary skill in the art, the specific meanings of the above terms in the present disclosure can be understood according to specific circumstances.

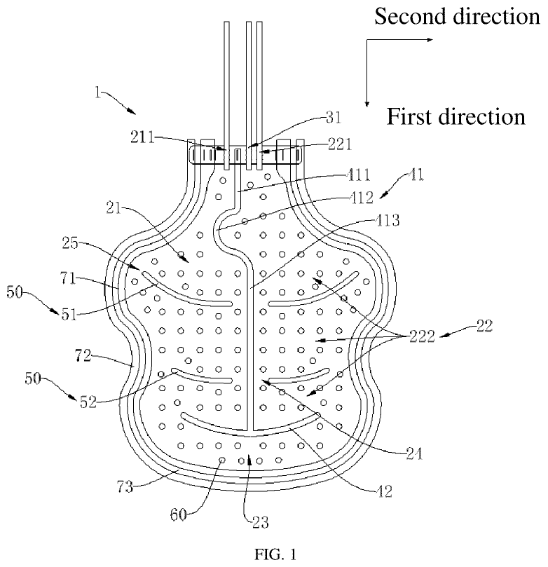

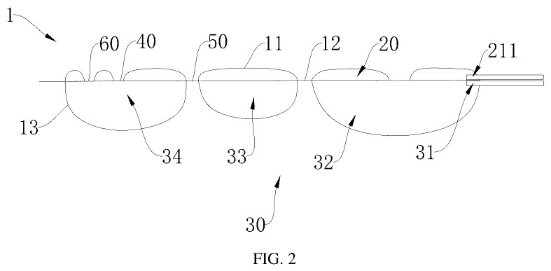

A cold compress bag 1 is provided. As shown in FIG. 1 to FIG. 6, the cold compress bag 1 includes a first surface layer 11, an intermediate layer 12, a second surface layer 13, and a partition assembly 40. A liquid storage space 20 is defined between the first surface layer 11 and the intermediate layer 12. An air storage space 30 is defined between the second surface layer 13 and the intermediate layer 12. The air storage space 30 is provided with a vent port 31. The vent port 31 is configured to communicate the air storage space 30 with outside. The partition assembly 40 is configured to connect to the first surface layer 11 and the intermediate layer 12 to divide the liquid storage space 20 into a liquid inlet region 21, and a liquid outlet region 22 in communication with the liquid inlet region 21. The liquid inlet region 21 is provided with a liquid inlet port 211 configured to communicate the liquid inlet region 21 with outside. The liquid outlet region 22 is provided with a liquid outlet port 221 configured to communicate the liquid outlet region 22 with outside.

It should be understood that the air storage space 30 of the cold compress bag 1 may be inflated or deflated through the vent port 31, so that when the cold compress bag 1 is bound to a limb of a patient, the cold compress bag 1 can intermittently apply pressure to the limb of the patient, to provide pneumatic compression therapy to prevent venous thrombosis. Further, liquid sequentially passes through the liquid inlet region 21 and the liquid outlet region 22 through the liquid inlet port 211 and the liquid outlet port 221, and circulates between the cold compress bag 1 and a cold compress device 2, to uniformly and continuously cold compress the limb of the patient to alleviate tissue swelling, bleeding, pain, and inflammatory reaction.

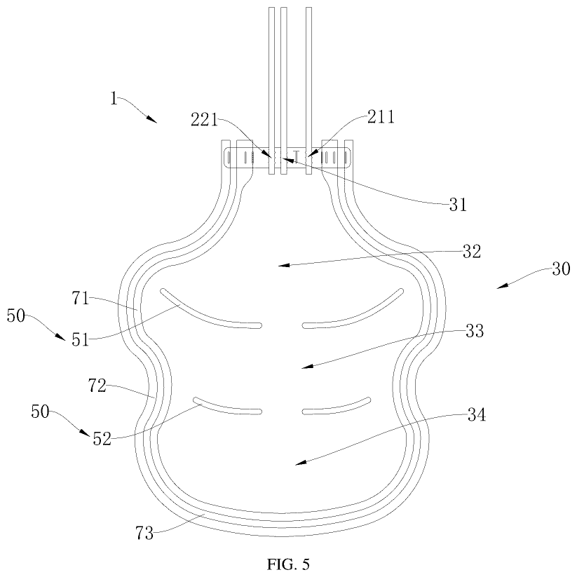

In some embodiments, as shown in FIG. 5, the cold compress bag 1 further includes a plurality of guide portions 50 configured to connect to the second surface layer 13 and the intermediate layer 12 to divide the air storage space 30 along a first direction into a first clamping region 32, a retaining region 33, and a second clamping region 34 in communication with one another. Specifically, the vent port 31 and the retaining region 33 are respectively located on opposing sides of the first clamping region 32, so that air can enter the first clamping region 32, the retaining region 33, and the second clamping region 34 in sequence through the vent port 31.

It should be understood that due to the blocking by the guide portions 50, air can quickly fill up the first clamping region 32 first, then gradually fill up the retaining region 33, and then gradually fill up the second clamping region 34. In other words, the first clamping region 32, the retaining region 33, and the second clamping region 34 are inflated in sequence, so that a region-wise gradual compression and decompression effect can be achieved, thereby providing pneumatic compression therapy of the limb of the patient. It should be noted that if the cold compress bag 1 is directly filled up, the limb will be subjected to pressure from many different directions, which is not conducive to guiding blood circulation. In this embodiment, the cold compress bag 1 can achieve a region-wise gradual compression and decompression effect to promote the blood circulation in a certain direction, thereby better promoting the flow of blood and lymph.

It should be noted that, compared with a straight-through air storage space 30 in which no guide portion 50 is provided, the arrangement of the guide portions 50 in this embodiment allows the first clamping region 32, the retaining region 33, and the second clamping region 34 to expand in sequence to achieve a region-wise compression effect, which is conducive to simulating a natural muscle pumping action to better prevent venous thrombosis. Further, region-wise compression is conducive to adjusting the pressure according to the specific condition of the patient to improve the comfort and effectiveness of pneumatic compression therapy.

In addition, the arrangement of the guide portions 50 can improve the bendability of the cold compress bag 1. In other words, the cold compress bag 1 is bendable at the guide portions 50, which is conducive to making the first clamping region 32, the retaining region 33, and the second clamping region 34 better fit a limb of a patient, thereby achieving a more comprehensive and snug wrap, improving the comfort of use, and improving the operability and convenience of binding the cold compress bag 1 to a limb of a patient by medical staff. It should be understood that when pneumatic compression therapy and cold therapy are carried out at the same time, the improved bendability and conformability of the cold compress bag 1 are conducive to making the cold compress bag 1 better fit the skin of the patient, thereby improving the effect of cold therapy.

In some embodiments, as shown in FIG. 1 to FIG. 4, the guide portions 50 are configured to connect to the first surface layer 11 and the intermediate layer 12, the guide portions 50 are spaced apart from an outer edge 70 of the cold compress bag 1 to define an outer passage 25 between the guide portions 50 and the outer edge 70 of the cold compress bag 1, and the guide portions 50 are spaced apart from the partition assembly 40 to define an inner passage 24 between the guide portions 50 and the partition assembly 40. In other words, the guide portions 50 are configured to connect to the first surface layer 11, the intermediate layer 12, and the second surface layer 13, and the guide portions 50 not only divide the air storage space 30 along the first direction into the first clamping region 32, the retaining region 33, and the second clamping region 34, but also divide the liquid inlet region 21 of the liquid storage space 20 along the first direction into a plurality of liquid inlet chambers 212 in communication with each other and divide the liquid outlet region 22 along the first direction into a plurality of liquid outlet chambers 222 in communication with each other. It should be understood that the guide portions 50 are spaced apart from the outer edge 70 so that the liquid flows from the outer passage 25 between the guide portions 50 and the outer edge 70 to the liquid inlet chambers 212 and the liquid outlet chambers 222, and the guide portions 50 are spaced apart from the partition assembly 40 so that the liquid flows from the inner passage 24 between the guide portions 50 and the partition assembly 40 to the liquid inlet chambers 212 and the respective liquid outlet chambers 222.

If the guide portions 50 are connected to the outer edge 70 or if the guide portions 50 are connected to the partition assembly 40, dead zones and vortexes may be formed at the connection. In this embodiment, the liquid can flow in the outer passage 25 and the inner passage 24, which is conducive to reducing dead zones and vortexes in the liquid storage space 20 and improving the flow efficiency and heat exchange efficiency of the liquid, allowing the liquid to circulate more smoothly between the cold compress bag 1 and the cold compress device 2.

In addition, when the liquid needs to be completely emptied, the arrangement of the outer passage 25 and the inner passage 24 can reduce dead zones between the guide portions 50, the partition assembly 40, and the outer edge 70, so that the liquid can be more smoothly evacuated from the cold compress bag 1, and the amount of residual liquid in the cold compress bag 1 can be reduced, thereby reducing the growth of bacteria and improving the hygiene.

It should be noted that, the first surface layer 11, the intermediate layer 12, and the second surface layer 13 can all be bent at the guide portions 50, which is conducive to further improving the bendability and conformability of the cold compress bag 1. In addition, the arrangement of the guide portions 50 can also enhance the strength of connection between the first surface layer 11, the intermediate layer 12, and the second surface layer 13, thereby improving the structural reliability of the cold compress bag 1 and reducing the risk of an edge portion of the cold compress bag 1 being ruptured by air or liquid.

In some embodiments, as shown in FIG. 1 to FIG. 4, the partition assembly 40 includes a primary partition 41 and a secondary partition 42. The primary partition 41 is configured to divide the liquid storage space 20 along the second direction into the liquid inlet region 21 and the liquid outlet region 22, the secondary partition 42 is connected to the primary partition 41, and an extending direction of the secondary partition 42 forms an angle relative to an extending direction of the primary partition 41 to define a transition passage 23 on a side away from the liquid inlet port 211 and the liquid outlet port 221. The transition passage 23 is configured to communicate the liquid inlet region 21 with the liquid outlet region 22. It should be understood that the liquid enters the liquid storage space 20 through the liquid inlet port 211, flows through the liquid inlet chambers 212 of the liquid inlet region 21, the transition passage 23, and the liquid outlet chambers 222 of the liquid outlet region 22 in sequence, and is then discharged out of the liquid storage space 20 through the liquid outlet port 221, i.e., the liquid can complete approximately one full circuit in the liquid storage space 20.

Without the arrangement of the partition assembly 40 to divide the liquid storage space 20 into the liquid inlet region 21, the transition passage 23, and the liquid outlet region 22, the liquid may mainly flow on a side adjacent to the liquid inlet port 211 and the liquid outlet port 221, and it is difficult for the liquid on the side away from the liquid inlet port 211 and the liquid outlet port 221 to flow sufficiently and participate in the circulation between the cold compress bag 1 and the cold compress device 2. In this embodiment, the arrangement of the partition assembly 40 allows the liquid to flow through the liquid inlet region 21, the transition passage 23 and the liquid outlet region 22 in an orderly manner to complete approximately one full circuit in the liquid storage space 20, to achieve a more uniform temperature distribution throughout the cold compress bag 1 while ensuring efficient liquid circulation, thereby reducing the risk of skin frostbite caused by prolonged unduly low temperature in a local area and the risk of ineffective cold therapy caused by prolonged unduly high temperature in a local area, and improving the comfort of the patient and the reliability of the effect of cold therapy.

In at least one embodiment, as shown in FIG. 1 to FIG. 7, the liquid inlet port 211, the liquid outlet port 221, and the vent port 31 are located on the same side of the outer edge 70 of the cold compress bag 1. As such, when the liquid inlet port 211, the liquid outlet port 221, and the vent port 31 are respectively in communication with the cold compress device 2 through pipes, the pipes can be integrated into one pipe bundle and thus be conveniently connected to another pipe bundle connected to the cold compress device 2, thereby improving the convenience of operation.

It should be noted that as shown in FIG. 1, the primary partition 41 is arranged adjacent to a centerline of the liquid storage space 20 extending in the first direction, so that the volume difference between the liquid inlet region 21 and the liquid outlet region 22 is small; and the liquid inlet port 211 and the liquid outlet port 221 are respectively located on two sides of the primary partition 41, so that the amount of liquid in the liquid inlet region 21 is kept substantially consistent with that in the liquid outlet region 22, thereby further improving the cooling effect.

In some embodiments, as shown in FIG. 1, the primary partition 41 includes a first segment 411, a transition segment 412, and a second segment 413, the first segment 411 is staggered with the second segment 413 in the second direction, and the transition segment 412 extends in a curved manner and is configured to connect to the first segment 411 and the second segment 413, so that from a side adjacent to the liquid inlet port 211 to a side away from the liquid inlet port 211 in the first direction, a width of at least a part of the liquid inlet region 21 in the second direction gradually increases.

It should be understood that, the liquid has a large flow rate and pressure when flowing from the cold compress device 2 to the cold compress bag 1, and the widening of at least a part of the flow passage of the liquid inlet region 21 can reduce the flow rate of the liquid that enters the liquid storage space 20, to reduce the wear and impact on the first surface layer 11, the intermediate layer 12, and the outer edge 70 of the cold compress bag 1 caused by the liquid, thereby improving the durability of the cold compress bag 1, prolonging the service life of the cold compress bag 1, and reducing the risk of leakage caused by the cold compress bag 1 being ruptured by the liquid. In addition, reducing the flow rate can also reduce the pressure loss of the liquid and allow to the liquid to flow more stably, thereby reducing the risk of irritation of the skin receiving cold therapy caused by the impact force of the liquid flow, and improving the comfort of the patient.

Furthermore, compared with a transition segment 412 that extends in a straight line or in a bent manner to connect the first segment 411 and the second segment 413, in this embodiment, the transition segment 412 extending in a curved manner provides a smooth transition between the first segment 411 and the second segment 413 to guide the flow of the liquid, and is conducive to avoiding the formation of corners, reducing dead zones, and reducing obstruction of the flow of the liquid by the primary partition 41, thereby improving the efficiency of liquid circulation and the efficiency of emptying the liquid.

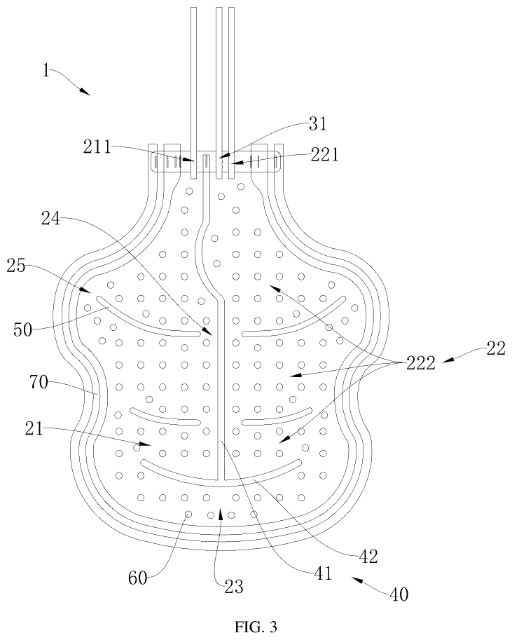

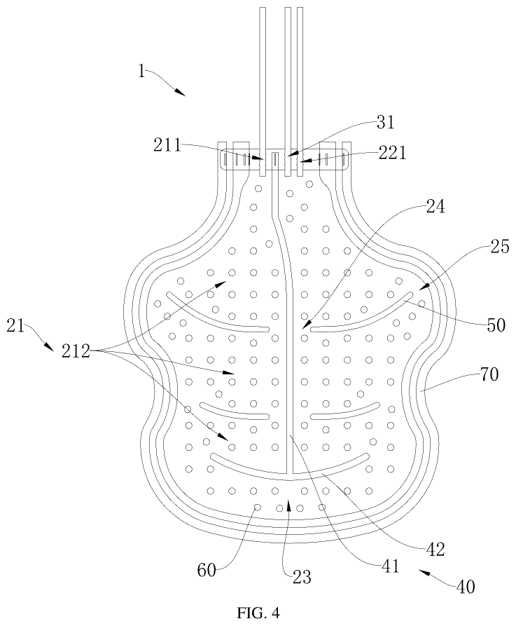

In at least one embodiment, as shown in FIG. 1, the transition segment 412 is in a form of one or more circular arcs. In at least one other embodiment, as shown in FIG. 3 and FIG. 4, the transition segment 412 is in a form of a parabola, a hyperbola, an elliptic arc, a sinusoidal curve, an exponential curve, an irregular curve, or the like. This is not particularly limited herein.

In at least one embodiment, as shown in FIG. 1 to FIG. 4, the second segment 413 is arranged adjacent to a centerline of the cold compress bag 1 to evenly divide the liquid storage space 20 into the liquid inlet region 21 and the liquid outlet region 22, so that the liquid pressure in the liquid storage space 20 remains stable, and the liquid can flow more stably and smoothly, thereby improving the efficiency of liquid circulation and the efficiency of emptying the liquid. The first segment 411 is offset in the second direction toward the liquid inlet region 21, so that the first segment 411 is staggered from the second segment 413 in the second direction, and from the side adjacent to the liquid inlet port 211 to the side away from the liquid inlet port 211 in the first direction, a width of a part of the liquid inlet region 21 corresponding to the transition segment 412 in the second direction gradually increases, providing a pressure relief effect. It should be noted that along the first direction, the transition segment 412 is located adjacent to the side of the cold compress bag 1 provided with the liquid inlet port 211, so that a pressure relief effect can be provided in a timely manner after the liquid enters the liquid inlet region 21 of the liquid storage space 20.

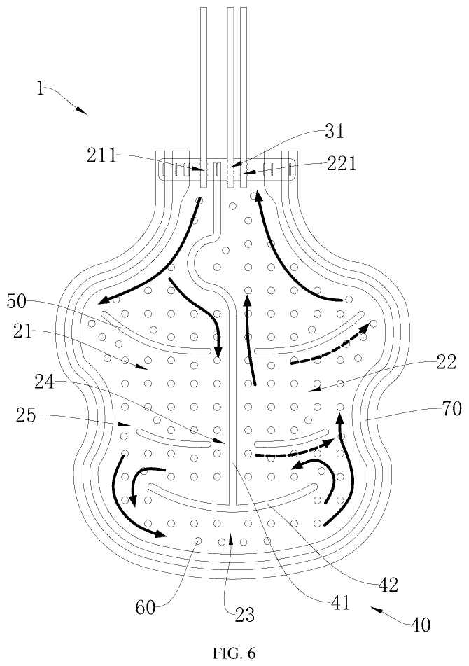

In some embodiments, as shown in FIG. 1 to FIG. 4, the secondary partition 42 extends in a curved manner in the second direction, and a center of curvature of the secondary partition 42 is located on a side facing the liquid inlet port 211 and the liquid outlet port 221. It should be understood that the secondary partition 42 and the outer edge 70 on the side of the cold compress bag 1 away from the liquid inlet port 211 are spaced apart from each other in the first direction, so that the transition passage 23 is formed between the secondary partition 42 and the outer edge 70. The secondary partition 42 is arranged closer to the liquid inlet port 211 than the outer edge 70 on the side away from the liquid inlet port 211 is, and the secondary partition 42 extends in the second direction to block the liquid. In other words, the liquid in the liquid inlet region 21 needs to bypass the secondary partition 42 to enter the transition passage 23, as denoted by arrowed solid lines in FIG. 6. This prevents the liquid from directly impacting the outer edge 70 in the first direction, thereby further reducing the risk of leakage caused by the cold compress bag 1 being ruptured by the liquid.

Further, because the center of curvature of the secondary partition 42 is located on the side facing the liquid inlet port 211 and the liquid outlet port 221, the secondary partition 42 can guide the liquid to flow toward the liquid outlet region 22 when the liquid needs to be emptied, to prevent the liquid from staying in the transition passage 23, thereby improving the efficiency of emptying the liquid, reducing the amount of residual liquid in the cold compress bag 1, and reducing the growth of bacteria.

In at least one embodiment, as shown in FIG. 1 to FIG. 4, the secondary partition 42 is arranged parallel to or approximately parallel to a part of the outer edge 70 opposite to the secondary partition 42 in the first direction, so that the transition passage 23 between the secondary partition 42 and the outer edge 70 has a uniform width in the first direction, which allows the liquid to flow through the transition passage 23 more stably and smoothly and reduces the generation of turbulence, thereby further improving the efficiency of liquid circulation and the efficiency of emptying the liquid.

It should be noted that, the primary partition 41 is configured to extend in the first direction to connect the first surface layer 11 and the intermediate layer 12, and the secondary partition 42 is configured to extend in the second direction to connect the first surface layer 11 and the intermediate layer 12, so that the strength of connection between the first surface layer 11 and the intermediate layer 12 can be improved, thereby improving the structural reliability of the cold compress bag 1.

In some embodiments, as shown in FIG. 1 and FIG. 5, the guide portions 50 include a first guide portion 51 and a second guide portion 52. The first guide portion 51 and the second guide portion 52 extend in a curved manner in the second direction. A center of curvature of the first guide portion 51 and a center of curvature of the second guide portion 52 are each located on a side facing the liquid inlet port 211 and the liquid outlet port 221. The first guide portion 51 is arranged closer to the liquid inlet port 211 and the liquid outlet port 221 than the second guide portion 52 is. A length of the first guide portion 51 is greater than a length of the second guide portion 52.

It should be understood that, because the first guide portion 51 is arranged closer to the liquid inlet port 211 and the liquid outlet port 221 than the second guide portion 52 is, and the length of the first guide portion 51 is greater than the length of the second guide portion 52, the outer passage 25 between the first guide portion 51 and the outer edge 70 has a small width, the first guide portion 51 can bear a large part of the impact force of the liquid in the first direction, and the liquid can be guide to the inner passage 24 between the first guide portion 51 and the partition assembly 40, as denoted by arrowed solid lines in FIG. 6. The outer passage 25 between the second guide portion 52 and the outer edge 70 has a large width, allowing the liquid to flow therethrough stably and smoothly.

Further, because the centers of curvature of the first guide portion 51 and the second guide portion 52 are located on the side facing the liquid inlet port 211 and the liquid outlet port 221, the first guide portion 51 and the second guide portion 52 can guide the liquid to flow toward the outer passage 25 when the liquid needs to be emptied, as denoted by arrowed dashed lines in FIG. 6. As such, the liquid can flow to the liquid outlet port 221 through the outer passage 25 and the transition passage 23, and the liquid is prevented from staying in the inner passage 24, the liquid inlet chambers 212, and the liquid outlet chambers 222, thereby improving the efficiency of emptying the liquid, reducing the amount of residual liquid in the cold compress bag 1, and reducing the growth of bacteria.

In at least one embodiment, as shown in FIG. 1 and FIG. 5, a set of first guide portion 51 and second guide portion 52 is provided on each of two sides of the primary partition 41 in the second direction, and from an end adjacent to the primary partition 41 to an end adjacent to the outer edge 70, the first guide portion 51 and the second guide portion 52 are curved toward the side where the liquid inlet port 211 and the liquid outlet port 221 are located, to guide the liquid to the outer passage 25. As such, when the liquid needs to be emptied, the risk of the liquid being blocked by the first guide portion 51 and the second guide portion 52 in the first direction can be further reduced, so that the amount of residual liquid in the cold compress bag 1 is further reduced.

In some embodiments, as shown in FIG. 1 to FIG. 4, the cold compress bag 1 further includes a plurality of shunt portions 60, and at least a subset of the plurality of shunt portions 60 are arranged in an array to shunt a liquid in the liquid storage space 20. It should be understood that without the arrangement of the shunt portions 60, the flow rate of the liquid near the center of the liquid storage space 20 will differ greatly from the flow rates of the liquid near the outer edge 70, the guide portions 50, and the partition assembly 40, leading to a high possibility of generation of turbulence. In this embodiment, the arrangement of the shunt portions 60 can increase the friction area between the liquid and the cold compress bag 1 and reduce the differences between the flow rates of the liquid at different positions in the liquid storage space 20, thereby avoiding the generation of turbulence by the liquid.

It should be noted that, the plurality of shunt portions 60 are configured to connect to the first surface layer 11 and the intermediate layer 12, to improve the strength of connection between the first surface layer 11 and the intermediate layer 12, thereby improving the structural reliability of the cold compress bag 1. Further, with the arrangement of the shunt portions 60, the thickness of the cold compress bag 1 filled up with the liquid can be reduced, so that the cold compress bag 1 filled up with the liquid can also be easily rolled up and spread. In addition, the arrangement of the shunt portions 60 is conducive to improving the bendability and conformability of the cold compress bag 1, making the cold compress bag 1 better fit the skin of a patient, thereby improving the effect of cold therapy.

In some embodiments, as shown in FIG. 1 to FIG. 6, the outer edge 70 of the cold compress bag 1 includes a plurality of smoothly connected arc segments configured to guide a flow direction of the liquid in the liquid storage space 20. It should be understood that compared with an outer edge 70 extending in a bent manner, the outer edge 70 in this embodiment is in a form of a plurality of smoothly connected arc segments, which is conducive to reducing the resistance of the outer edge 70 to the flow of the liquid, thereby reducing the pressure loss and improving the stability of the flow of the liquid. Further, the outer edge 70 in the form of the plurality of smoothly connected arc segments is conducive to reducing dead zones and vortexes, so that when the liquid needs to be emptied, the risk of the liquid staying in a dead zone at a corner of the outer edge 70 is reduced, thereby further reducing the amount of residual liquid in the cold compress bag 1.

In addition, the continuous guiding effect of the outer edge 70 on the flow direction of the liquid is also conducive to reducing the impact force of the liquid on the outer edge 70 and achieving a more uniform stress distribution on the outer edge 70, thereby reducing the risk of leakage of the cold compress bag 1 caused by the outer edge 70 being ruptured by the liquid.

Further, as shown in FIG. 1, the arc segments include two concave segments 72, and each of the concave segments 72 is curved inwardly toward a center of the cold compress bag 1. When the cold compress bag 1 is bound to a limb of a patient, the concave segments 72 are configured to avoid a joint, such as elbow, elbow socket, wrist, knee, popliteal socket, ankle, etc., so as not to affect the normal motion of the patient, making the cold compress bag 1 more ergonomic to improve the comfort of the patient using the cold compress bag 1.

In at least one embodiment, as shown in FIG. 1, the outer edge 70 further includes two first convex segments 71 and two second convex segments 73, and each of the concave segments 72 is configured to connect to the corresponding first convex segment 71 and the second convex segment 73. Specifically, the outer edge 70 of the cold compress bag 1 has a symmetrical structure along the second direction, and the first convex segments 71 are curved outwardly away from the center of the cold compress bag 1 to cooperate with the transition segment 412 of the primary partition 41 to provide a pressure relief effect in a timely manner after the liquid enters the liquid inlet region 21 of the liquid storage space 20, and guide the liquid to the liquid outlet port 221, thereby improving the efficiency of emptying the liquid, as denoted by arrowed solid lines in FIG. 6. The concave segments 72 are arranged opposite to the retaining region 33 of the air storage space 30 in the second direction, so that the cold compress bag 1 can avoid a joint when bound to a limb of a patient. The second convex segment 73 extends in a curved manner to connect to the two concave segments 72. Further, the second convex segment 73 is arranged opposite to the secondary partition 42 in the first direction, so that the transition passage 23 is formed between the second convex segment 73 and the secondary partition 42. It should be understood that the second convex segment 73 extending in a curved manner is conducive to guiding the liquid to flow from the liquid inlet region 21 to the transition passage 23 and from the transition passage 23 to the liquid outlet region 22, allowing the liquid to flow more smoothly in the liquid storage space 20, thereby improving the efficiency of liquid circulation and the efficiency of emptying the liquid.

In some embodiments, when the liquid needs to be emptied, the cold compress device 2 may pump the liquid from the liquid storage space 20, and at the same time, the cold compress device 2 may inflate the air storage space 30 to increase the air pressure and thus squeeze the cold compress bag 1, simulating an action of squeezing the cold compress bag 1 with a hand to allow the liquid in the liquid storage space 20 to flow. This facilitates the flow of the liquid back to the cold compress device 2, so that the amount of residual liquid in the cold compress bag 1 can be further reduced.

It should be understood that, when the cold compress device 2 inflates the air storage space 30, the intermediate layer 12 and the second surface layer 13 are stretched apart by the air storage space 30, the intermediate layer 12 provides a squeezing force toward the first surface layer 11 to drive the liquid to flow in the liquid storage space 20, and at the same time, the partition assembly 40, the guide portions 50, the shunt portions 60, and the outer edge 70 provide guidance to the liquid, to prevent the liquid from staying in dead zones and enable the liquid to be more fully discharged out of the liquid storage space.

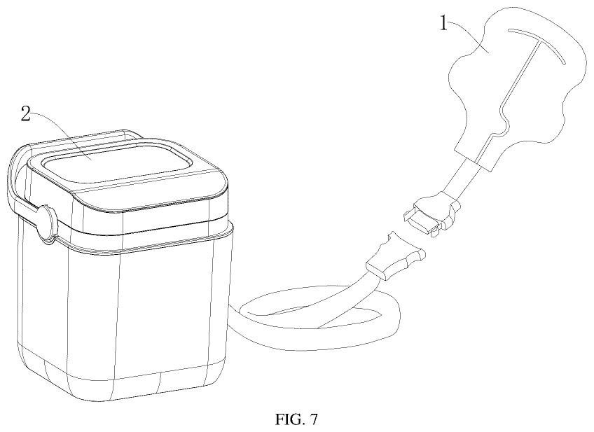

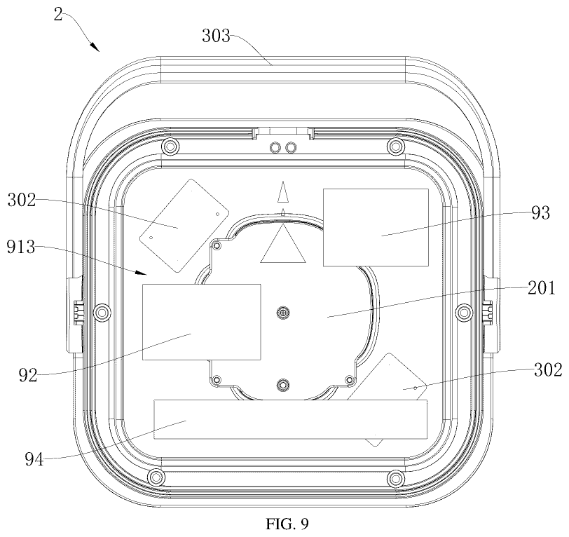

A cold compress system is provided. As shown in FIG. 7 to FIG. 13, the cold compress system includes the cold compress bag 1 described above and a cold compress device 2.

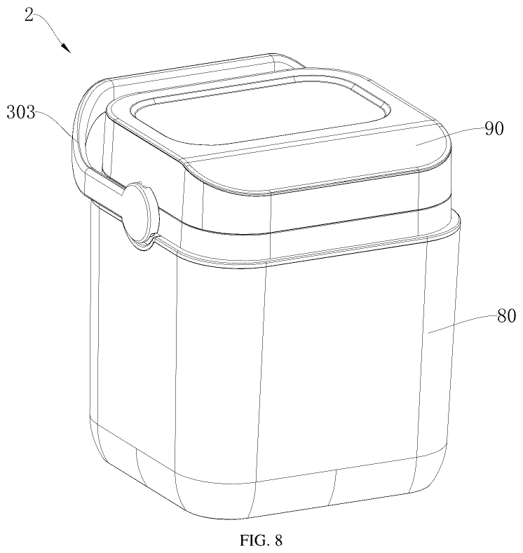

As shown in FIG. 8 to FIG. 10, the cold compress device 2 includes a box body 80, a box lid 90, and a liquid diversion structure 101. The box body 80 is provided with an accommodating cavity 81 configured to accommodate liquid. The box lid 90 is configured to cover a top of the box body 80. The box lid 90 includes a lid body 91, a liquid intake and drainage module 92, a pressurizing module 93, and a control module 94. The lid body 91 is provided with an accommodating space 913 configured to accommodate the liquid intake and drainage module 92, the pressurizing module 93, and the control module 94. The liquid intake and drainage module 92 is configured to circulate the liquid between the box body 80 and the cold compress bag 1 to allow the cold compress device 2 to operate in a cold therapy mode. The liquid intake and drainage module 92 is configured to pump the liquid from the cold compress bag 1 to allow the cold compress device 2 to operate in a drainage mode. The pressurizing module 93 is configured to inflate the cold compress bag 1 to allow the cold compress device 2 to operate in a pressurizing mode. The control module 94 is configured to control the liquid intake and drainage module 92 and the pressurizing module 93 to allow the cold compress device 2 to operate in at least one of the pressurizing mode, the cold therapy mode, and the drainage mode. The liquid diversion structure 101 extends from the lid body 91 to a bottom of the box body 80. The liquid diversion structure 101 is configured to communicate the accommodating cavity 81 with the liquid intake and drainage module 92.

Specifically, the liquid intake and drainage module 92 is in communication with the liquid inlet port 211 of the cold compress bag 1 through at least one inlet pipe, so that the liquid flows from the cold compress device 2 to the cold compress bag 1. The liquid intake and drainage module 92 is in communication with the liquid outlet port 221 of the cold compress bag 1 through at least one return pipe, so that the liquid flows back from the cold compress bag 1 to the cold compress device 2. The pressurizing module 93 is in communication with the vent port 31 of the cold compress bag 1 through at least one air pipe to inflate the cold compress bag 1.

It should be understood that the cold compress device 2 may operate in the cold therapy mode by the control module 94 and the liquid intake and drainage module 92, the liquid intake and drainage module 92 circulates the liquid between the cold compress device 2 and the cold compress bag 1, and the cold compress bag 1 is bound to a limb of a patient to cool the limb of the patient, thereby alleviating tissue swelling, bleeding, pain, and inflammatory reaction.

Further, the cold compress device 2 may operate in the pressurizing mode by the control module 94 and the pressurizing module 93, the pressurizing module 93 inflates and expands the cold compress bag 1, and the cold compress bag 1 is bound to a limb of a patient to apply pressure to the limb, thereby preventing venous thrombosis, enhancing venous blood flow of lower limbs, and so on.

Furthermore, the cold compress device 2 may operate in the drainage mode by the control module 94 and the liquid intake and drainage module 92, and the liquid intake and drainage module 92 discharges the liquid from the cold compress bag 1, to prevent the liquid from staying in the cold compress bag 1, thereby reducing the growth of bacteria and improving the hygiene.

It should be noted that, compared with a case where a liquid discharge port is provided on the cold compress bag 1 and the liquid in the cold compress bag 1 is discharged manually, in this embodiment, the liquid intake and drainage module 92 is used to discharge the liquid from the cold compress bag 1, which is conducive to reducing the workload of medical staff and facilitates the operation of the patient. In addition, this obviates the need to provide a liquid discharge port on the cold compress bag 1, so that the sealing performance of the cold compress bag 1 is improved.

In some embodiments, when the cold compress device 2 operates in the drainage mode, the pressurizing module 93 inflates the cold compress bag 1 to squeeze the cold compress bag 1, which facilitates the flow of the liquid back to the cold compress device 2, thereby further reducing the amount of residual liquid in the cold compress bag 1.

In at least one embodiment, in the early stage of the drainage mode, only the liquid intake and drainage module 92 operates to quickly discharge the liquid from the cold compress bag 1; and in the later stage of the drainage mode, the liquid intake and drainage module 92 and the pressurizing module 93 operate simultaneously, and the pressurizing module 93 inflates the cold compress bag 1 to squeeze the liquid in the cold compress bag 1 toward the return pipe, so that the drainage efficiency is improved.

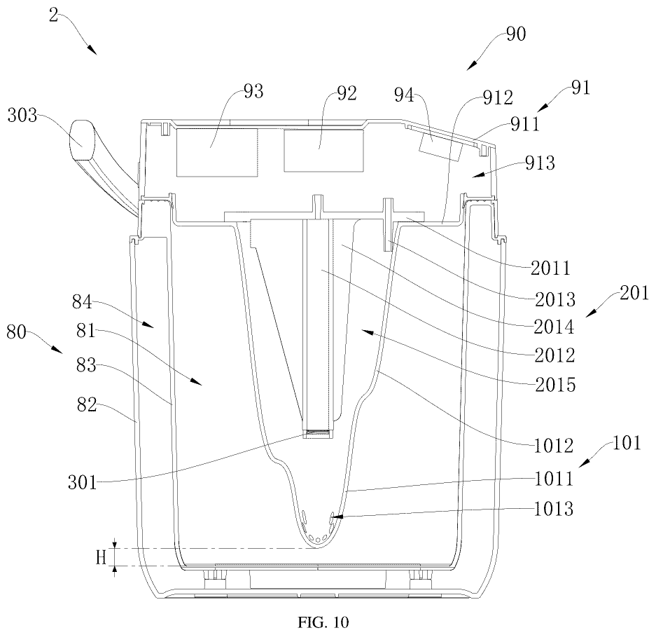

In some embodiments, as shown in FIG. 10 to FIG. 12, the liquid diversion structure 101 has a conical shape with a large top and a small bottom along an extending direction thereof, allowing liquid droplets on the surface of the liquid diversion structure 101 to easily slide off the surface, thereby reducing the formation of stains and scale on the surface of the liquid diversion structure 101 and improving the hygiene of the cold compress device 2. In addition, the conical structure with a large top and a small bottom is easy to clean. It should be understood that compared with a straight cylindrical liquid diversion structure 101, the liquid diversion structure 101 in this embodiment facilitates the insertion of a cleaning tool, so that the liquid diversion structure 101 can be cleaned more conveniently and thoroughly.

In some embodiments, as shown in FIG. 10 to FIG. 12, the liquid diversion structure 101 includes a main body 1011 and a plurality of outer arc portions 1012 surrounding an outer periphery of the main body 1011. The plurality of outer arc portions 1012 protrude from the main body 1011 in a radial direction, making the liquid diversion structure 101 have a conical shape with a large top and a small bottom. It should be understood that an arc surface at a position where an end of each of the outer arc portions 1012 is connected to the main body 1011 forms a small angle with a horizontal plane, making it easy for liquid droplets to directly drip from the ends of the outer arc portions 1012, thereby further reducing the formation of stains and scale on the surface of the liquid diversion structure 101. In addition, a radial dimension of the outer arc portion 1012 is larger than that of the main body 1011, which is conducive to improving the structural strength of the liquid diversion structure 101. It should be noted that the main body 1011 and the plurality of outer arc portions 1012 are configured so that the liquid diversion structure 101 resembles an iceberg, so that aesthetics of the liquid diversion structure 101 is improved.

In at least one embodiment, the plurality of outer arc portions 1012 are each of an irregular shape, and are different from each other. In at least one other embodiment, the outer arc portions 1012 are each in a form of an ellipsoid surface, an elliptic paraboloid surface, a single-leaf hyperboloid surface, a double-leaf hyperboloid surface, or the like, which is not particularly limited herein.

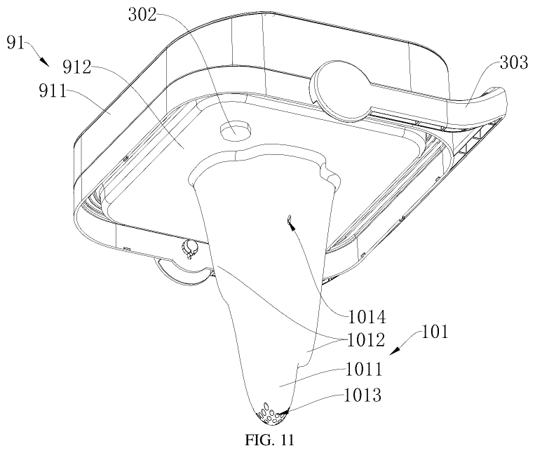

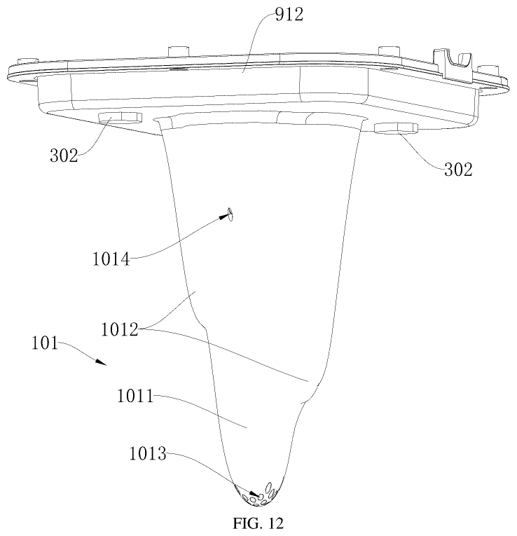

In some embodiments, as shown in FIG. 11 and FIG. 12, the liquid diversion structure 101 is provided with a plurality of inlet holes 1013 and at least one outlet hole 1014. The plurality of inlet holes 1013 are provided at an end of the liquid diversion structure 101 and are arranged opposite to the bottom of the box body 80. The liquid in the accommodating cavity 81 enters the liquid intake and drainage module 92 through the inlet holes 1013. The at least one outlet hole 1014 is located above the inlet holes 1013. The liquid is discharged from the liquid intake and drainage module 92 to the accommodating cavity 81 through the at least one outlet hole 1014.

In other words, the inlet holes 1013 are arranged adjacent to the bottom of the box body 80, so that the liquid intake and drainage module 92 can pump the liquid from a bottom layer of the accommodating cavity 81 into the cold compress bag 1. The outlet hole 1014 is located above the inlet holes 1013, i.e., arranged adjacent to the box lid 90 or adjacent to a liquid surface, so that the liquid discharged from the cold compress bag 1 flows to an upper layer of the accommodating cavity 81, i.e., flow to the liquid surface or to a position adjacent to the liquid surface. It should be understood that the temperature of the liquid pumped into the cold compress bag 1 is low, the temperature of the liquid discharged from the cold compress bag 1 is high, and as the discharged liquid flows to the liquid surface, the high-temperature liquid will not quickly transfer heat to the liquid adjacent to the bottom layer of the accommodating cavity 81, so that the liquid at the bottom layer of the accommodating cavity 81 can be kept at a low temperature.

In some embodiments, as shown in FIG. 10, a distance H from the end of the liquid diversion structure 101 to the bottom of the box body 80 is less than 5 cm. It should be understood that the inlet holes 1013 are provided at the end of the liquid diversion structure 101, i.e., the inlet holes 1013 are adjacent to the bottom of the box body 80. It should be understood that because the liquid pressure near the bottom of the box body 80 is higher than that at a position near the liquid surface, the arrangement of the inlet holes 1013 adjacent to the bottom of the box body 80 is conducive to improving the liquid suction efficiency and reducing the disturbance to the liquid surface during the liquid suction process, so that the liquid can be sucked into the cold compress bag 1 more stably. It should be noted that the arrangement of the inlet holes 1013 adjacent to the bottom of the box body 80 can also prevent air from being sucked into the cold compress bag 1, thereby further improving the reliability of the cold compress device 2 and the cold compress bag 1 during use.

Preferably, the distance H from the end of the liquid diversion structure 101 to the bottom of the box body 80 is less than 3 cm. More preferably, the distance H from the end of the liquid diversion structure 101 to the bottom of the box body 80 is less than or equal to 1 cm.

In some embodiments, as shown in FIG. 10, the lid body 91 includes an upper lid 911 and a lower lid 912 buckled to the upper lid 911, and the accommodating space 913 is formed between the upper lid 911 and the lower lid 912 to accommodate the liquid intake and drainage module 92, the pressurizing module 93, and the control module 94. Further, the liquid diversion structure 101 is integrally formed on the lower lid 912. As such, the number of parts and the manufacturing costs are reduced, and the sealing performance between the liquid diversion structure 101 and the lower lid 912 is improved, so that the risk of the liquid entering the modules in the lid body 91 is reduced, and the reliability and safety of the cold compress device 2 are improved.

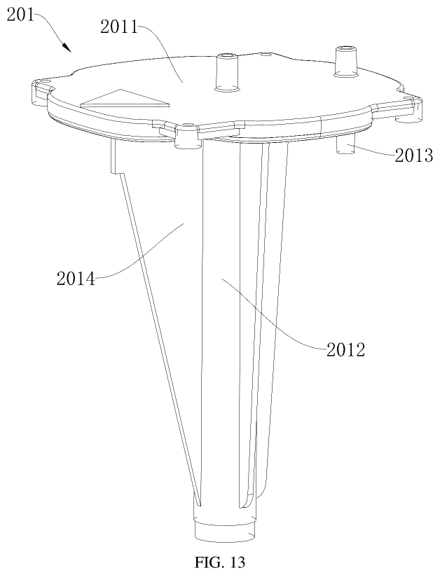

In some embodiments, as shown in FIG. 10 and FIG. 13, the cold compress device 2 further includes a pipe assembly 201. The pipe assembly 201 is arranged in the liquid diversion structure 101. The pipe assembly 201 includes a plate 2011, a primary pipe 2012, and a secondary pipe 2013. The plate 2011 is sealedly connected to the liquid diversion structure 101. A liquid diversion spacing 2015 is defined between the pipe assembly 201 and the sealed structure. The primary pipe 2012 extends from the plate 2011 toward the end of the liquid diversion structure 101 to communicate the liquid diversion spacing 2015 with the liquid intake and drainage module 92, allowing the liquid to enter the liquid intake and drainage module 92. The secondary pipe 2013 is located above the primary pipe 2012. The secondary pipe 2013 extends from the plate 2011 toward a side wall of the liquid diversion structure 101 to communicate the liquid diversion spacing 2015 with the liquid intake and drainage module 92, allowing the liquid to be discharged into the accommodating cavity 81.

It should be understood that under the action of the liquid intake and drainage module 92, the liquid in the accommodating cavity 81 enters the cold compress bag 1 through the inlet holes 1013 of the liquid diversion structure 101, the liquid diversion spacing 2015, the primary pipe 2012 of the pipe assembly 201, and the inlet pipe in sequence, and flows back to the accommodating cavity 81 through a return pipe of the pipe assembly 201, the secondary pipe 2013, the liquid diversion spacing 2015, and the outlet hole 1014 of the liquid diversion structure 101 in sequence.

In at least one embodiment, as shown in FIG. 10, the plate 2011 is fixed to the lower lid 912, and a sealing member is provided between the plate 2011 and the lower lid 912 to sealedly connect the pipe assembly 201 and the lower lid 912.

In at least one embodiment, as shown in FIG. 10 and FIG. 13, the pipe assembly 201 further includes a plurality of reinforcing ribs 2014 configured to connect to the plate 2011 and the primary pipe 2012, to improve the structural strength of the pipe assembly 201.

In at least one embodiment, the secondary pipe 2013 is arranged opposite to the outlet hole 1014 of the liquid diversion structure 101, so that the liquid discharged from the cold compress bag 1 enters the accommodating cavity 81 through the outlet hole 1014.

In some embodiments, as shown in FIG. 10, the cold compress device 2 further includes a filter member 301. The filter member 301 is mounted in the primary pipe 2012 to filter impurities, to prevent impurities from entering the liquid intake and drainage module 92 and the cold compress bag 1, thereby improving the reliability of operation of the cold compress device 2, and prolonging the service life of components such as a pump and a valve of the liquid intake and drainage module 92.

In some embodiments, the pressurizing module 93 includes an inflation and deflation assembly configured to inflate and deflate the cold compress bag 1 and an air pressure sensing element configured to detect an air pressure in the cold compress bag 1. It should be understood that by the cooperation of the inflation and deflation assembly and the air pressure sensing element, the cold compress bag 1 can intermittently apply pressure to a limb to simulate the blood vessel pressure during natural movement. For example, when the cold compress bag 1 is bound to a calf of a patient, the pressure intermittently applied by the cold compress bag 1 can simulate the blood vessel pressure during natural walking, to massage muscles and prevent venous thrombosis. It should be noted that the air pressure sensing element can detect the pressure in the cold compress bag 1, and the operation of the inflation and deflation assembly can be adjusted to control the pressure more precisely, to prevent the pressure from being unduly high to cause ischemia and hypoxia or unduly low to affect the therapeutic effect.

In at least one embodiment, the pressurizing module 93 is configured to control the pressure between 35 mmHg and 40 mmHg.

In at least one embodiment, the inflation and deflation assembly includes an air pump and a solenoid valve. The air pump is configured to inflate the cold compress bag 1. The solenoid valve is configured to deflate the cold compress bag 1.

In some embodiments, the liquid intake and drainage module 92 includes a liquid pump and a valve. The liquid pump is configured to circulate the liquid between the cold compress device 2 and the cold compress bag 1. The valve is configured to block the liquid from entering the cold compress bag 1. The liquid pump pumps the liquid from the cold compress bag 1 to allow the cold compress device 2 to operate in the drainage mode. In other words, the liquid intake and drainage module 92 can pump the liquid out of the cold compress bag 1. It should be understood that the liquid in the cold compress bag 1 may also be discharged by other means, which is not particularly limited herein.

In some embodiments, as shown in FIG. 10, the box body 80 includes an outer shell 82 and an inner shell 83, the outer shell 82 is sleeved over an outer periphery of the inner shell 83, and an isolation spacing 84 is defined between the outer shell 82 and the inner shell 83. It should be understood that the outer shell 82 and the inner shell 83 spaced apart from each other can provide a heat insulation effect, to reduce the heat exchange between an ice-liquid mixture in the box body 80 and the outside, so that the ice-liquid ratio in the ice-liquid mixture in the box body 80 can be kept in a range of 1:1.4 to 1:2.5 for an extended period of time.

In at least one embodiment, as shown in FIG. 10 and FIG. 12, the isolation spacing 84 is evacuated to vacuum. In at least one other embodiment, the isolation spacing 84 is filled with inert gas, air, gel, etc., which is not particularly limited herein.

In some embodiments, the outer shell 82 and the inner shell 83 of the box body 80 are each made of a transparent material, and the cold compress device 2 further includes a lighting lamp 302 configured to illuminate the accommodating cavity 81 of the box body 80, to facilitate observation of the ice-liquid mixture in the box body 80 in a dark environment.

In at least one embodiment, the lighting lamp 302 is configured to change color depending on different operational modes, allowing a user to quickly determine the operational mode of the cold compress device 2.

In some embodiments, as shown in FIG. 8 to FIG. 11, the cold compress device 2 further includes a handle 303 rotatably connected to the box body 80 or the box lid 90 to facilitate movement and transportation of the cold compress device 2.

The basic principles, main features and advantages of the present disclosure have been described above. Those skilled in the art should understand that the present disclosure is not limited to the above-mentioned embodiments. The descriptions of the embodiments and the specification are only for illustrating the principles of the present disclosure. Various changes and improvements may be made to the present disclosure without departing from the spirit and scope of the present disclosure. and such changes and improvements all fall within the scope of protection claimed by the present disclosure. The scope of protection claimed by the present disclosure is defined by the appended claims and equivalents thereof.

1. A cold compress bag, comprising:
a first surface layer, an intermediate layer, and a second surface layer, wherein a liquid storage space is defined between the first surface layer and the intermediate layer, an air storage space is defined between the second surface layer and the intermediate layer, the air storage space is provided with a vent port, and the vent port is configured to communicate the air storage space with outside;
a partition assembly, wherein the partition assembly is configured to connect to the first surface layer and the intermediate layer to divide the liquid storage space into a liquid inlet region, and a liquid outlet region in communication with the liquid inlet region, the liquid inlet region is provided with a liquid inlet port configured to communicate the liquid inlet region with the outside, and the liquid outlet region is provided with a liquid outlet port configured to communicate the liquid outlet region with the outside; and
wherein the partition assembly comprises a primary partition and a secondary partition, the primary partition is configured to divide the liquid storage space along a second direction into the liquid inlet region and the liquid outlet region, the secondary partition is connected to the primary partition, an extending direction of the secondary partition forms an angle relative to an extending direction of the primary partition, the secondary partition extends in a curved manner in the second direction to define a transition passage on a side away from the liquid inlet port and the liquid outlet port, and the transition passage is configured to communicate the liquid inlet region with the liquid outlet region; and
a plurality of guide portions, wherein the plurality of guide portions are configured to connect to the second surface layer and the intermediate layer to divide the air storage space along a first direction into a first clamping region, a retaining region, and a second clamping region in communication with one another, the plurality of guide portions are configured to connect to the first surface layer and the intermediate layer, the plurality of guide portions are spaced apart from an outer edge of the cold compress bag to define an outer passage between the plurality of guide portions and the outer edge of the cold compress bag, and the plurality of guide portions are spaced apart from the partition assembly to define an inner passage between the plurality of guide portions and the partition assembly; and the cold compress bag is bendable at the plurality of guide portions.

2. The cold compress bag according to claim 1, wherein the primary partition comprises a first segment, a transition segment, and a second segment; the first segment is staggered with the second segment in the second direction; the transition segment extends in a curved manner and is configured to connect to the first segment and the second segment; and from a side adjacent to the liquid inlet port to a side away from the liquid inlet port in the first direction, a width of at least a part of the liquid inlet region in the second direction gradually increases.

3. The cold compress bag according to claim 1, wherein a center of curvature of the secondary partition is located on a side facing the liquid inlet port and the liquid outlet port.

4. The cold compress bag according to claim 1, wherein the plurality of guide portions comprise a first guide portion and a second guide portion, the first guide portion and the second guide portion extend in a curved manner in the second direction, a center of curvature of the first guide portion and a center of curvature of the second guide portion are each located on a side facing the liquid inlet port and the liquid outlet port, the first guide portion is arranged closer to the liquid inlet port and the liquid outlet port than the second guide portion is, and a length of the first guide portion is greater than a length of the second guide portion.

5. The cold compress bag according to claim 1, further comprising a plurality of shunt portions, wherein at least a subset of the plurality of shunt portions are arranged in an array to shunt a liquid in the liquid storage space.

6. The cold compress bag according to claim 1, wherein the outer edge of the cold compress bag comprises a plurality of arc segments smoothly connected, the plurality of arc segments are configured to guide a flow direction of a liquid in the liquid storage space, the plurality of arc segments comprise a concave segment, the concave segment is curved inwardly toward a center of the cold compress bag, and when the cold compress bag is bound to a limb, the concave segment is configured to avoid a joint.

7. A cold compress system, comprising:
the cold compress bag according to claim 1; and
a cold compress device, comprising:
a box body, wherein the box body is provided with an accommodating cavity configured to accommodate a liquid;
a box lid, wherein the box lid is configured to cover a top of the box body, the box lid comprises a lid body, a liquid intake and drainage module, a pressurizing module, and a control module, and the lid body is provided with an accommodating space configured to accommodate the liquid intake and drainage module, the pressurizing module, and the control module; the liquid intake and drainage module is configured to circulate the liquid between the box body and the cold compress bag to allow the cold compress device to operate in a cold therapy mode; the liquid intake and drainage module is configured to evacuate the liquid from the cold compress bag to allow the cold compress device to operate in a drainage mode; the pressurizing module is configured to inflate the cold compress bag to allow the cold compress device to operate in a pressurizing mode; and the control module is configured to control the liquid intake and drainage module and the pressurizing module to allow the cold compress device to operate in at least one of the pressurizing mode, the cold therapy mode, or the drainage mode; and
a liquid diversion structure, wherein the liquid diversion structure extends from the lid body to a bottom of the box body, and the liquid diversion structure is configured to communicate the accommodating cavity with the liquid intake and drainage module.

8. The cold compress bag according to claim 2, further comprising a plurality of shunt portions, wherein at least a subset of the plurality of shunt portions are arranged in an array to shunt a liquid in the liquid storage space.

9. The cold compress bag according to claim 3, further comprising a plurality of shunt portions, wherein at least a subset of the plurality of shunt portions are arranged in an array to shunt a liquid in the liquid storage space.

10. The cold compress bag according to claim 2, wherein the outer edge of the cold compress bag comprises a plurality of arc segments smoothly connected, the plurality of arc segments are configured to guide a flow direction of a liquid in the liquid storage space, the plurality of arc segments comprise a concave segment, the concave segment is curved inwardly toward a center of the cold compress bag, and when the cold compress bag is bound to a limb, the concave segment is configured to avoid a joint.

11. The cold compress bag according to claim 3, wherein the outer edge of the cold compress bag comprises a plurality of arc segments smoothly connected, the plurality of arc segments are configured to guide a flow direction of a liquid in the liquid storage space, the plurality of arc segments comprise a concave segment, the concave segment is curved inwardly toward a center of the cold compress bag, and when the cold compress bag is bound to a limb, the concave segment is configured to avoid a joint.

12. A cold compress system, comprising:
the cold compress bag according to claim 2; and
a cold compress device, comprising:
a box body, wherein the box body is provided with an accommodating cavity configured to accommodate a liquid;
a box lid, wherein the box lid is configured to cover a top of the box body, the box lid comprises a lid body, a liquid intake and drainage module, a pressurizing module, and a control module, and the lid body is provided with an accommodating space configured to accommodate the liquid intake and drainage module, the pressurizing module, and the control module; the liquid intake and drainage module is configured to circulate the liquid between the box body and the cold compress bag to allow the cold compress device to operate in a cold therapy mode; the liquid intake and drainage module is configured to evacuate the liquid from the cold compress bag to allow the cold compress device to operate in a drainage mode; the pressurizing module is configured to inflate the cold compress bag to allow the cold compress device to operate in a pressurizing mode; and the control module is configured to control the liquid intake and drainage module and the pressurizing module to allow the cold compress device to operate in at least one of the pressurizing mode, the cold therapy mode, or the drainage mode; and
a liquid diversion structure, wherein the liquid diversion structure extends from the lid body to a bottom of the box body, and the liquid diversion structure is configured to communicate the accommodating cavity with the liquid intake and drainage module.

13. A cold compress system, comprising:
the cold compress bag according to claim 3; and
a cold compress device, comprising:
a box body, wherein the box body is provided with an accommodating cavity configured to accommodate a liquid;
a box lid, wherein the box lid is configured to cover a top of the box body, the box lid comprises a lid body, a liquid intake and drainage module, a pressurizing module, and a control module, and the lid body is provided with an accommodating space configured to accommodate the liquid intake and drainage module, the pressurizing module, and the control module; the liquid intake and drainage module is configured to circulate the liquid between the box body and the cold compress bag to allow the cold compress device to operate in a cold therapy mode; the liquid intake and drainage module is configured to evacuate the liquid from the cold compress bag to allow the cold compress device to operate in a drainage mode; the pressurizing module is configured to inflate the cold compress bag to allow the cold compress device to operate in a pressurizing mode; and the control module is configured to control the liquid intake and drainage module and the pressurizing module to allow the cold compress device to operate in at least one of the pressurizing mode, the cold therapy mode, or the drainage mode; and
a liquid diversion structure, wherein the liquid diversion structure extends from the lid body to a bottom of the box body, and the liquid diversion structure is configured to communicate the accommodating cavity with the liquid intake and drainage module.

14. A cold compress system, comprising:
the cold compress bag according to claim 4; and
a cold compress device, comprising:
a box body, wherein the box body is provided with an accommodating cavity configured to accommodate a liquid;
a box lid, wherein the box lid is configured to cover a top of the box body, the box lid comprises a lid body, a liquid intake and drainage module, a pressurizing module, and a control module, and the lid body is provided with an accommodating space configured to accommodate the liquid intake and drainage module, the pressurizing module, and the control module; the liquid intake and drainage module is configured to circulate the liquid between the box body and the cold compress bag to allow the cold compress device to operate in a cold therapy mode; the liquid intake and drainage module is configured to evacuate the liquid from the cold compress bag to allow the cold compress device to operate in a drainage mode; the pressurizing module is configured to inflate the cold compress bag to allow the cold compress device to operate in a pressurizing mode; and the control module is configured to control the liquid intake and drainage module and the pressurizing module to allow the cold compress device to operate in at least one of the pressurizing mode, the cold therapy mode, or the drainage mode; and
a liquid diversion structure, wherein the liquid diversion structure extends from the lid body to a bottom of the box body, and the liquid diversion structure is configured to communicate the accommodating cavity with the liquid intake and drainage module.

15. A cold compress system, comprising:
the cold compress bag according to claim 5; and
a cold compress device, comprising:
a box body, wherein the box body is provided with an accommodating cavity configured to accommodate the liquid;
a box lid, wherein the box lid is configured to cover a top of the box body, the box lid comprises a lid body, a liquid intake and drainage module, a pressurizing module, and a control module, and the lid body is provided with an accommodating space configured to accommodate the liquid intake and drainage module, the pressurizing module, and the control module; the liquid intake and drainage module is configured to circulate the liquid between the box body and the cold compress bag to allow the cold compress device to operate in a cold therapy mode; the liquid intake and drainage module is configured to evacuate the liquid from the cold compress bag to allow the cold compress device to operate in a drainage mode; the pressurizing module is configured to inflate the cold compress bag to allow the cold compress device to operate in a pressurizing mode; and the control module is configured to control the liquid intake and drainage module and the pressurizing module to allow the cold compress device to operate in at least one of the pressurizing mode, the cold therapy mode, or the drainage mode; and
a liquid diversion structure, wherein the liquid diversion structure extends from the lid body to a bottom of the box body, and the liquid diversion structure is configured to communicate the accommodating cavity with the liquid intake and drainage module.

16. A cold compress system, comprising:
the cold compress bag according to claim 6; and
a cold compress device, comprising:
a box body, wherein the box body is provided with an accommodating cavity configured to accommodate the liquid;
a box lid, wherein the box lid is configured to cover a top of the box body, the box lid comprises a lid body, a liquid intake and drainage module, a pressurizing module, and a control module, and the lid body is provided with an accommodating space configured to accommodate the liquid intake and drainage module, the pressurizing module, and the control module; the liquid intake and drainage module is configured to circulate the liquid between the box body and the cold compress bag to allow the cold compress device to operate in a cold therapy mode; the liquid intake and drainage module is configured to evacuate the liquid from the cold compress bag to allow the cold compress device to operate in a drainage mode; the pressurizing module is configured to inflate the cold compress bag to allow the cold compress device to operate in a pressurizing mode; and the control module is configured to control the liquid intake and drainage module and the pressurizing module to allow the cold compress device to operate in at least one of the pressurizing mode, the cold therapy mode, or the drainage mode; and
a liquid diversion structure, wherein the liquid diversion structure extends from the lid body to a bottom of the box body, and the liquid diversion structure is configured to communicate the accommodating cavity with the liquid intake and drainage module.

17. A cold compress system, comprising:
the cold compress bag according to claim 8; and
a cold compress device, comprising:
a box body, wherein the box body is provided with an accommodating cavity configured to accommodate the liquid;
a box lid, wherein the box lid is configured to cover a top of the box body, the box lid comprises a lid body, a liquid intake and drainage module, a pressurizing module, and a control module, and the lid body is provided with an accommodating space configured to accommodate the liquid intake and drainage module, the pressurizing module, and the control module; the liquid intake and drainage module is configured to circulate the liquid between the box body and the cold compress bag to allow the cold compress device to operate in a cold therapy mode; the liquid intake and drainage module is configured to evacuate the liquid from the cold compress bag to allow the cold compress device to operate in a drainage mode; the pressurizing module is configured to inflate the cold compress bag to allow the cold compress device to operate in a pressurizing mode; and the control module is configured to control the liquid intake and drainage module and the pressurizing module to allow the cold compress device to operate in at least one of the pressurizing mode, the cold therapy mode, or the drainage mode; and
a liquid diversion structure, wherein the liquid diversion structure extends from the lid body to a bottom of the box body, and the liquid diversion structure is configured to communicate the accommodating cavity with the liquid intake and drainage module.

18. A cold compress system, comprising:
the cold compress bag according to claim 9; and
a cold compress device, comprising:
a box body, wherein the box body is provided with an accommodating cavity configured to accommodate the liquid;
a box lid, wherein the box lid is configured to cover a top of the box body, the box lid comprises a lid body, a liquid intake and drainage module, a pressurizing module, and a control module, and the lid body is provided with an accommodating space configured to accommodate the liquid intake and drainage module, the pressurizing module, and the control module; the liquid intake and drainage module is configured to circulate the liquid between the box body and the cold compress bag to allow the cold compress device to operate in a cold therapy mode; the liquid intake and drainage module is configured to evacuate the liquid from the cold compress bag to allow the cold compress device to operate in a drainage mode; the pressurizing module is configured to inflate the cold compress bag to allow the cold compress device to operate in a pressurizing mode; and the control module is configured to control the liquid intake and drainage module and the pressurizing module to allow the cold compress device to operate in at least one of the pressurizing mode, the cold therapy mode, or the drainage mode; and
a liquid diversion structure, wherein the liquid diversion structure extends from the lid body to a bottom of the box body, and the liquid diversion structure is configured to communicate the accommodating cavity with the liquid intake and drainage module.

19. A cold compress system, comprising:
the cold compress bag according to claim 10; and
a cold compress device, comprising:
a box body, wherein the box body is provided with an accommodating cavity configured to accommodate the liquid;
a box lid, wherein the box lid is configured to cover a top of the box body, the box lid comprises a lid body, a liquid intake and drainage module, a pressurizing module, and a control module, and the lid body is provided with an accommodating space configured to accommodate the liquid intake and drainage module, the pressurizing module, and the control module; the liquid intake and drainage module is configured to circulate the liquid between the box body and the cold compress bag to allow the cold compress device to operate in a cold therapy mode; the liquid intake and drainage module is configured to evacuate the liquid from the cold compress bag to allow the cold compress device to operate in a drainage mode; the pressurizing module is configured to inflate the cold compress bag to allow the cold compress device to operate in a pressurizing mode; and the control module is configured to control the liquid intake and drainage module and the pressurizing module to allow the cold compress device to operate in at least one of the pressurizing mode, the cold therapy mode, or the drainage mode; and
a liquid diversion structure, wherein the liquid diversion structure extends from the lid body to a bottom of the box body, and the liquid diversion structure is configured to communicate the accommodating cavity with the liquid intake and drainage module.

20. A cold compress system, comprising:
the cold compress bag according to claim 11; and
a cold compress device, comprising:
a box body, wherein the box body is provided with an accommodating cavity configured to accommodate the liquid;
a box lid, wherein the box lid is configured to cover a top of the box body, the box lid comprises a lid body, a liquid intake and drainage module, a pressurizing module, and a control module, and the lid body is provided with an accommodating space configured to accommodate the liquid intake and drainage module, the pressurizing module, and the control module; the liquid intake and drainage module is configured to circulate the liquid between the box body and the cold compress bag to allow the cold compress device to operate in a cold therapy mode; the liquid intake and drainage module is configured to evacuate the liquid from the cold compress bag to allow the cold compress device to operate in a drainage mode; the pressurizing module is configured to inflate the cold compress bag to allow the cold compress device to operate in a pressurizing mode; and the control module is configured to control the liquid intake and drainage module and the pressurizing module to allow the cold compress device to operate in at least one of the pressurizing mode, the cold therapy mode, or the drainage mode; and
a liquid diversion structure, wherein the liquid diversion structure extends from the lid body to a bottom of the box body, and the liquid diversion structure is configured to communicate the accommodating cavity with the liquid intake and drainage module.
